(19) United States
(12) Patent Application Publication
(10) Pub. No.: US 2025/0011321 A1
(43) Pub. Date: Jan. 9, 2025

(54) Nonlinear Optical Chromophores with Indolizine Donor Groups

(71) Applicant: Lightwave Logic, Inc., Englewood, CO (US)

(72) Inventors: Ginelle A. Ramann, Centennial, CO (US); Barry L. Johnson, Castle Rock, CO (US); Cory Pecinovsky, Lafayette, CO (US)

(73) Assignee: Lightwave Logic, Inc., Englewood, CO (US)

(21) Appl. No.: 18/756,051

(22) Filed: Jun. 27, 2024

Related U.S. Application Data
(60) Provisional application No. 63/524,047, filed on Jun. 29, 2023.

(51) Int. Cl. C07D 471/04

(52) U.S. Cl. CPC C07D 471/04

(57) ABSTRACT

The present invention is directed, in general, to nonlinear optical chromophores having an indolizine donor group. Various embodiments of the present invention include nonlinear optical chromophores with indolizine donor group having an indolizine donor connected to a II-bridge group. In various preferred embodiments of the present invention, the indolizine donor groups may be substituted or unsubstituted, including hydro and alkyl substituents, aryl substituents and combination thereof. In certain embodiments of the present invention, the substituent moieties in the indolizine donor groups may be important for isolation. The isolation may reduce chromophore-chromophore interaction. The isolation may reduce charge transfer between molecules. The isolation may increase maximum applied voltage, which may contribute to more optimal poling and higher r₃₃.

### CROSS-REFERENCE TO RELATED APPLICATIONS

This application claims the benefit of U.S. Provisional Application No. 63/524,047 filed on Jun. 29, 2023, the entire contents of which is incorporated herein by reference.

### BACKGROUND

Nonlinear optical (NLO) chromophores provide the electro-optic (EO) activity in poled, electro-optic polymer devices. Electro-optic polymers have been investigated for many years as an alternative to inorganic materials such as lithium niobate in electro-optic devices. Electro-optic devices may include, for example, external modulators for telecom, datacom, RF photonics, and optical interconnects and so forth. Polymeric electro-optic materials have demonstrated enormous potential for core application in a broad range of next-generation systems and devices, including electro-optic modulators, optical switches, phased array radar, satellite and fiber telecommunications, cable television (CATV), optical gyroscopes for application in aerial and missile guidance, electronic counter measure (ECM) systems, backplane interconnects for high-speed computation, ultraquick analog-to-digital conversion, land mine detection, radio frequency photonics, spatial light modulation and all-optical (light-switching-light) signal processing.

Many NLO molecules (chromophores) have been synthesized that exhibit high molecular electro-optic properties. The product of the molecular dipole moment (μ) and hyperpolarizability (β) is often used as a measure of molecular electro-optic performance due to the dipole's involvement in material processing. See Dalton et al., “New Class of High Hyperpolarizability Organic Chromophores and Process for Synthesizing the Same”, WO 00/09613.

Nevertheless, extreme difficulties have been encountered translating microscopic molecular hyperpolarizabilities (β) into macroscopic material hyperpolarizabilities (χ²). Molecular subcomponents (chromophores) must be integrated into NLO materials that exhibit (i) a high degree of macroscopic nonlinearity and (ii) sufficient temporal, thermal, chemical and photochemical stability. High electro-optic activity and the stability of electro-optic activity, which is also referred to as “temporal stability,” are important for commercially viable devices. Electro-optic activity may be increased in electro-optic polymers by increasing the concentration of nonlinear optical chromophores in a host polymer and by increasing of the electro-optic property of chromophores. However, some techniques for increasing chromophore concentration may decrease poling efficiency and temporal stability. Simultaneous solution of these dual issues is regarded as the final impediment in the broad commercialization of EO polymers in numerous devices and systems.

The production of high material hyperpolarizabilities (χ²) is limited by the poor social character of NLO chromophores. Commercially viable materials must incorporate chromophores at large molecular densities with the requisite molecular moment statistically oriented along a single material axis. In order to achieve such an organization, the charge transfer (dipole) character of NLO chromophores is commonly exploited through the application of an external electric field during material processing that creates a localized lower-energy condition favoring noncentrosymmetric order. Unfortunately, even at moderate chromophore densities, molecules form multi-molecular dipolarly-bound (centrosymmetric) aggregates that cannot be dismantled via realistic field energies. To overcome this difficulty, integration of anti-social dipolar chromophores into a cooperative material architecture is commonly achieved through the construction of physical barriers (e.g., anti-packing steric groups) that limit proximal intermolecular relations.

Thus, it has often been considered advantageous in the art to produce nonlinear optical chromophore containing materials that exhibit a high glass transition temperature (T_{g}). Materials with a high glass transition temperature exhibit improved thermal stability and maintain their macroscopic electro-optic properties to a greater degree than materials with lower glass transition temperatures. It can also be advantageous to produce nonlinear optical chromophores which exhibit desirable macroscopic optical properties, thermal stability and have lower wavelength maximum absorption, i.e., λₘₐₓ.

### BRIEF SUMMARY

The present invention is directed, in general, to nonlinear optical chromophores having an indolizine donor group. Various embodiments of the present invention include nonlinear optical chromophores with indolizine donor group having an indolizine donor connected to a II-bridge group. In various embodiments of the present invention, the indolizine donor groups may be substituted or unsubstituted, including hydro and alkyl substituents, aryl substituents and combination thereof. In certain embodiments of the present invention, the substituent moieties in the indolizine donor groups may be important for isolation. The isolation may reduce chromophore-chromophore interaction. The isolation may reduce charge transfer between molecules. The isolation may increase maximum applied voltage, which may contribute to more optimal poling and higher r₃₃.

Various embodiments of the present invention include nonlinear optical chromophores having a C₈-C₂₀ bridging group between donor and acceptor. Various embodiments of the present invention include nonlinear optical chromophores having a C₄-C₆ bridging group between donor and acceptor, which chromophores exhibit lowered λₘₐₓ values. In certain embodiments, nonlinear optical chromophores have a C₄-C₆ bridging group between donor and acceptor, an R¹ moiety which can represent any alkyl group, and a R² moiety which can represent any aryl group. These nonlinear chromophores having a C₄-C₆ bridging group between donor and acceptor exhibit lowered λₘₐₓ values, can have narrow absorption bands. In certain other embodiments nonlinear optical chromophores are characterized by a Δλₘₐₓ based λₘₐₓ of chromophore analogs in a nonpolar solvent.

Amax values of the embodiments described herein create a larger transparency range among large wavelengths than do λₘₐₓ values outside of the ranges described herein. For example, various nonlinear optical chromophore embodiments provide transparency over 800 nm, which have applications in 1) short-distance local area networks, 2) free-space optical fiber communications and optical interconnection in conjunction with LED or semiconductor lasers, and 3) active materials for terahertz (THz) applications. Various embodiments of the present invention include nonlinear optical chromophores having a short chain bridging group between donor and acceptor, which chromophores exhibit decreased optical loss and can provide higher degree of transparency at various wavelengths. In addition, various embodiments of the present invention have optimized characteristics of (1) higher r₃₃ (electro-optic coefficients) at various wavelengths, (2) higher T_{g} (glass transition temperature), (3) exceptionally high thermal stability, and (4) high photostability.

Various embodiments of the present invention include a nonlinear optical chromophore of the general formula (I):

D-II-A (I)

wherein D represents an organic electron-donating group; A represents an organic electron-accepting group having an electron affinity greater than the electron affinity of D; and II represents a II-bridge between A and D; wherein the II-bridge comprises a carbon chain covalently bound to and separating A and D, wherein the carbon chain length between A and D is 2 to 4 carbon atoms, and wherein the 2 to 4 carbon atom chain comprises up to 2 carbon-carbon double bonds and up to 4 pendant substituents, wherein two or more of the pendant substituents can form a ring structure with the 2 to 4 carbon atom chain.

In various embodiments of the present invention, nonlinear optical chromophores of the general formula (I) have an indolizine donor group. In various embodiments of the present invention, nonlinear optical chromophores of the general formula (I) have an indolizine donor group connected to a II-bridge group.

Other aspects, features and advantages will be apparent from the following disclosure, including the detailed description, preferred embodiments, and the appended claims.

### BRIEF DESCRIPTION OF SEVERAL VIEWS OF THE DRAWINGS

The foregoing summary, as well as the following detailed description of preferred embodiments of the invention, will be better understood when read in conjunction with the appended drawings. For purposes of illustration the invention, there are shown in the drawings embodiments which are presently preferred. It should be understood, however, that the invention is not limited to the precise arrangements and instrumentalities shown.

In the drawings:

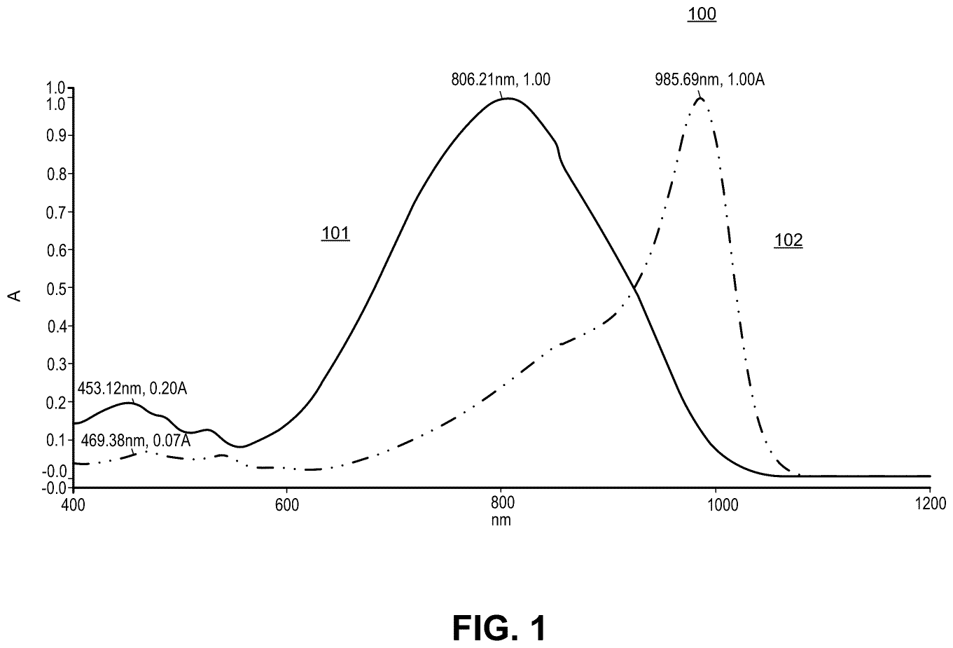

Spectrograph 100 of FIG. 1 illustrates the absorption of an example indolizine chromophore of general formula (I) in two different solvents over the UV-Vis spectrum, where first curve 101 shows the absorption of the example short chain bridge chromophore of general formula (I) in 1,4-dioxane solvent over the UV-Vis spectrum, whereas second curve 102 shows the absorption of the example short chain bridge chromophore of general formula (I) in dichloromethane (DCM) solvent over the UV-Vis spectrum.

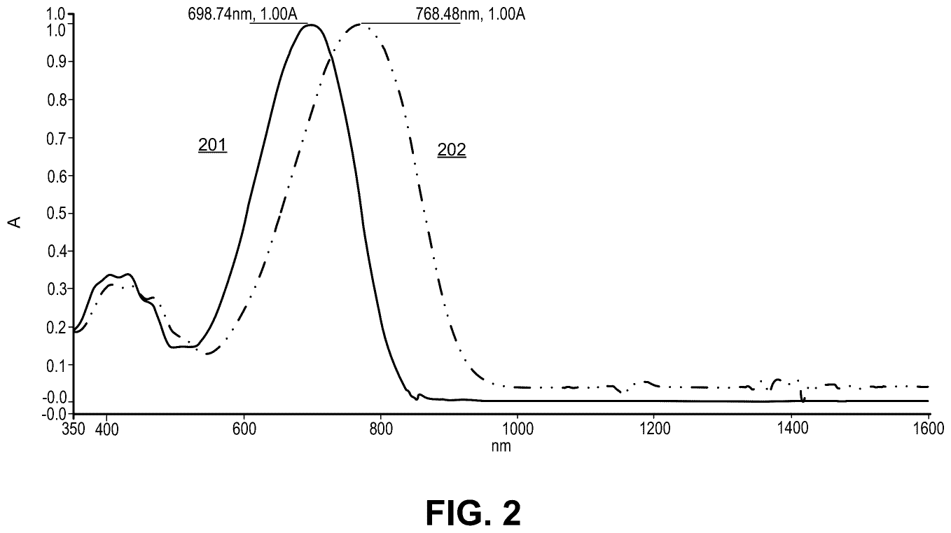

Spectrograph 200 of FIG. 2 illustrates the absorption of an example indolizine chromophore of general formula (I) in two different solvents over the UV-Vis spectrum, where first curve 201 shows the absorption of the example short chain bridge chromophore of general formula (I) in 1,4-dioxane solvent over the UV-Vis spectrum, whereas second curve 202 shows the absorption of the example short chain bridge chromophore of general formula (I) in dichloromethane (DCM) solvent over the UV-Vis spectrum.

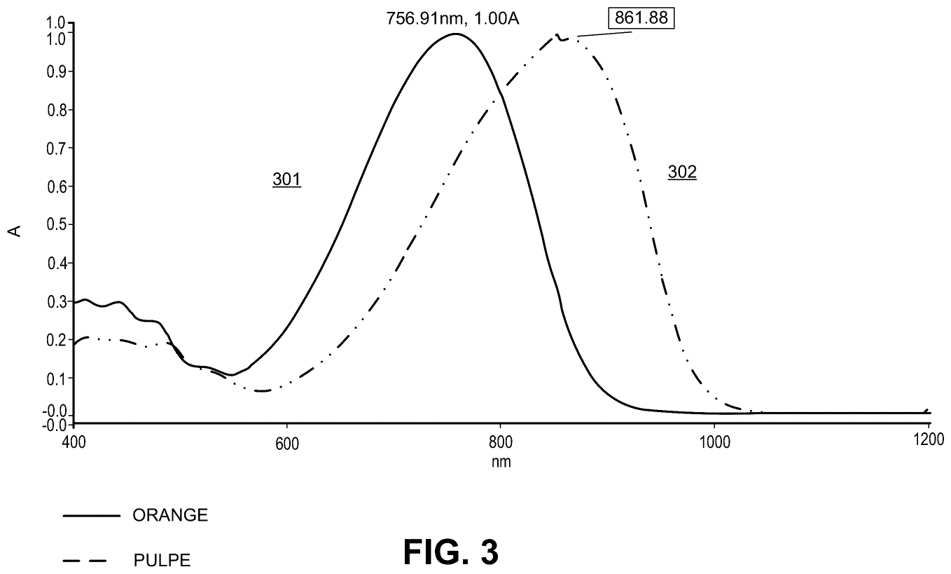

Spectrograph 300 of FIG. 3 illustrates the absorption of an example indolizine chromophore of general formula (I) in two different solvents over the UV-Vis spectrum, where first curve 301 shows the absorption of the example short chain bridge chromophore of general formula (I) in 1,4-dioxane solvent over the UV-Vis spectrum, whereas second curve 302 shows the absorption of the example short chain bridge chromophore of general formula (I) in dichloromethane (DCM) solvent over the UV-Vis spectrum.

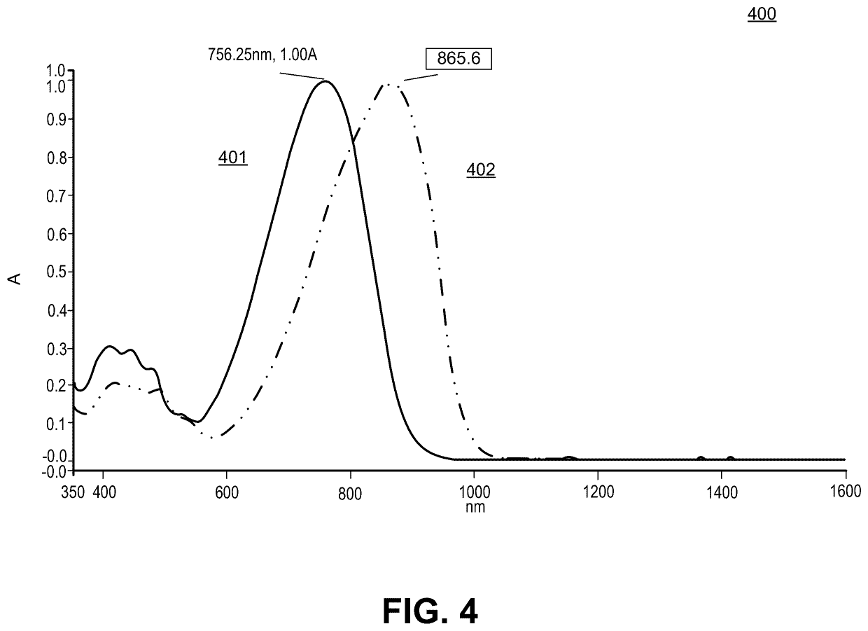

Spectrograph 400 of FIG. 4 illustrates the absorption of an example indolizine chromophore of general formula (I) in two different solvents over the UV-Vis spectrum, where first curve 401 shows the absorption of the example short chain bridge chromophore of general formula (I) in 1,4-dioxane solvent over the UV-Vis spectrum, whereas second curve 402 shows the absorption of the example short chain bridge chromophore of general formula (I) in dichloromethane (DCM) solvent over the UV-Vis spectrum.

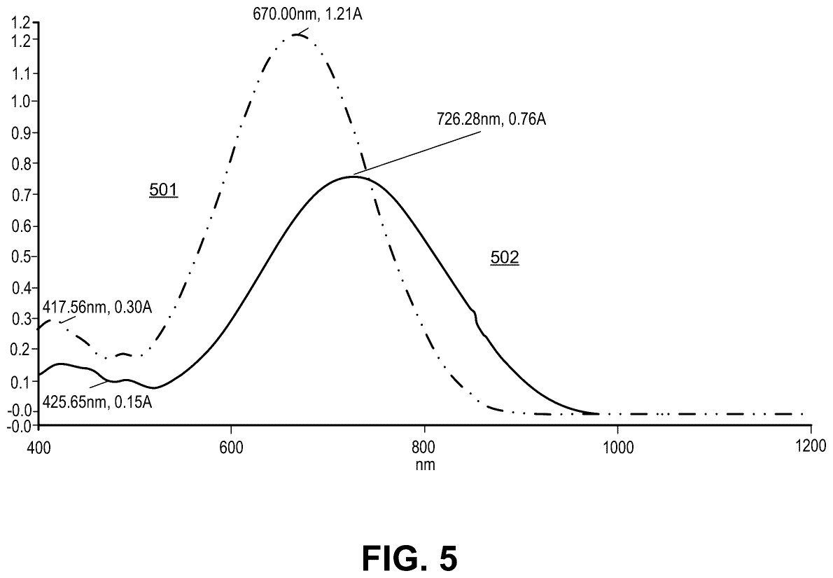

Spectrograph 500 of FIG. 5 illustrates the absorption of an example indolizine chromophore of general formula (I) in two different solvents over the UV-Vis spectrum, where first curve 501 shows the absorption of the example short chain bridge chromophore of general formula (I) in 1,4-dioxane solvent over the UV-Vis spectrum, whereas second curve 502 shows the absorption of the example short chain bridge chromophore of general formula (I) in dichloromethane (DCM) solvent over the UV-Vis spectrum.

### DETAILED DESCRIPTION

As used herein, the singular terms “a” and “the” are synonymous and used interchangeably with “one or more” and “at least one,” unless the language and/or context clearly indicates otherwise. Accordingly, for example, reference to “a polymer” or “the polymer” herein or in the appended claims can refer to a single polymer or more than one polymer. As a further example, reference to “a solvent” or “the solvent” herein or in the appended claims can refer to a single solvent or a mixture of more than one solvent. Additionally, all numerical values, unless otherwise specifically noted, are understood to be modified by the word “about.”

As used herein, the term “nonlinear optic chromophore” (NLOC) refers to molecules or portions of a molecule that create a nonlinear optic effect when irradiated with light. The chromophores are any molecular unit whose interaction with light gives rise to the nonlinear optical effect. The desired effect may occur at resonant or nonresonant wavelengths. The activity of a specific chromophore in a nonlinear optic material is stated as its hyper-polarizability, which is directly related to the molecular dipole moment of the chromophore. The various embodiments of NLO chromophores of the present invention are useful structures for the production of NLO effects.

The first-order hyperpolarizability (B) is one of the most common and useful NLO properties. Higher-order hyperpolarizabilities are useful in other applications such as all-optical (light-switching-light) applications. To determine if a material, such as a compound or polymer, includes a nonlinear optic chromophore with first-order hyperpolar character and a sufficient electro-optic coefficient (r₃₃), which is a function of β, the following test may be performed. First, the material in the form of a thin film is placed in an electric field to align the dipoles. This may be performed by sandwiching a film of the material between electrodes, such as indium tin oxide (ITO) substrates, gold films, or silver films, for example.

Nonlinear optical chromophores of embodiments herein have an indolizine electron-donating group and a C₆-C₈ chain bridging group between donor and acceptor, which chromophores have optimal Amax values and exhibit good electro-optic characters. The λₘₐₓ has to be in an optimal region for achieving good optimal electro-optic affect. For example, nonlinear optical chromophores having a bridging group between indolizine donor and acceptor can have a λₘₐₓ in DCM between 725 nm and 900 nm.

The Δλₘₐₓ is measured by calculating the λₘₐₓ of the nonlinear optic chromophore in a standard polar solvent (e.g., DCM) versus the λₘₐₓ an analog in a nonpolar solvent (e.g., 1,4-dioxane). The less-polar (1,4-dioxane) solvent will stabilize the neutral ground-state chromophore, while the more-polar solvent (DCM) will stabilize the charge-transfer state of the chromophore. The more easily the charge-transfer can happen, the more “polarizable”, and hence the more active the chromophore. Here, it has been found that higher wavelengths are directly proportional to lower energy. So, the higher Δλₘₐₓ between two solvents, the less energy is required to achieve the charge-transfer state. In addition, the r₃₃, which is measured experimentally and equates to the responsiveness of the light passing through to the electric field being applied, has a general correlation with the Δλₘₐₓ, wherein the larger Δλₘₐₓ generally correlates to a larger r₃₃. In that manner, the Δλₘₐₓ is characteristic of the “strength” of the chromophore's electro-optic activity.

Referring to FIG. 1, absorption of Chromophore (IB5822) was measured in two different solvents. λₘₐₓ is 985.7 nm in dichloromethane (DCM) and λₘₐₓ is 806.2 nm in 1,4-dioxane.

Referring to FIG. 2, absorption of Chromophore (IB5826) was measured in two different solvents. λₘₐₓ is 768.5 nm in dichloromethane (DCM) and λₘₐₓ is 698.7 nm in 1,4-dioxane.

Referring to FIG. 3, absorption of Chromophore (IB5829) was measured in two different solvents. λₘₐₓ is 861.9 nm in dichloromethane (DCM) and λₘₐₓ is 756.9 nm in 1,4-dioxane.

Referring to FIG. 4, absorption of Chromophore (IB5831) was measured in two different solvents. λₘₐₓ is 865.6 nm in dichloromethane (DCM) and λₘₐₓ is 756.3 nm in 1,4-dioxane.

Referring to FIG. 5, absorption of Chromophore (KO5201) was measured in two different solvents. λₘₐₓ is 726.3 nm in dichloromethane (DCM) and λₘₐₓ is 670 nm in 1,4-dioxane.

λₘₐₓ and Δλₘₐₓ data of short chain bridge chromophores of FIGS. 1-5 as well as other chromophores is summarized as follows:

| | | | |
|---|---|---|---|
| Chromophore | λₘₐₓ DCM | λₘₐₓ 1,4-dioxane | Δλₘₐₓ |

| | | | | | | |
|---|---|---|---|---|---|---|
| IL9041 | 870.9 | nm | 743.6 | nm | 127.3 | nm |
| IL9048 | 868 | nm | 757.8 | nm | 110.2 | nm |
| IB5805 | 773.9 | nm | 699.2 | nm | 74.7 | nm |
| IB5821 | 828.3 | nm | 738.1 | nm | 90.2 | nm |
| IB5822 | 985.7 | nm | 806.2 | nm | 179.5 | nm |
| IB5826 | 768.5 | nm | 698.7 | nm | 69.8 | nm |
| IB5829 | 861.9 | nm | 756.9 | nm | 105 | nm |
| IB5831 | 865.6 | nm | 756.3 | nm | 109.3 | nm |
| KO5201 | 726.3 | nm | 670 | nm | 56.3 | nm |

To generate a poling electric field, an electric potential is then applied to the electrodes while the material is heated to near its glass transition (T_{g}) temperature. After a suitable period of time, the temperature is gradually lowered while maintaining the poling electric field. Alternatively, the material can be poled by corona poling method, where an electrically charged needle at a suitable distance from the material film provides the poling electric field. In either instance, the dipoles in the material tend to align with the field. Various embodiments according to the present invention can include or can also include electro-optic materials having a material glass transition temperature greater than or equal to 100° C., greater than or equal to 125° C., or greater than or equal to 150° C., or even higher.

The nonlinear optical property of the poled material is then tested as follows. Polarized light, often from a laser, is passed through the poled material, then through a polarizing filter, and to a light intensity detector. If the intensity of light received at the detector changes as the electric potential applied to the electrodes varies, the material incorporates a nonlinear optic chromophore having an electro-optically variable refractive index. A more detailed discussion of techniques to measure the electro-optic constants of a poled film that incorporates nonlinear optic chromophores may be found in Chia-Chi Teng, Measuring Electro-Optic Constants of a Poled Film, in Nonlinear Optics of Organic Molecules and Polymers, Chp. 7, 447-49 (Hari Singh Nalwa & Seizo Miyata eds., 1997), incorporated by reference in its entirety, except that in the event of any inconsistent disclosure or definition from the present application, the disclosure or definition herein shall be deemed to prevail.

The relationship between the change in applied electric potential versus the change in the refractive index of the material may be represented as its electro-optical (OE) coefficient (r₃₃). This effect is commonly referred to as an electro-optic, or EO, effect. Devices that include materials that change their refractive index in response to changes in an applied electric potential are called electro-optical (EO) devices. For compositions having short chain bridge chromophores described herein, a high EO coefficient of >30 pm/V (measured, e.g., at 980 nm) or >20 pm/V (measured at, e.g., 1310 nm) can be achieved.

Compositions having short chain bridge chromophores described herein can also exhibit decreased optical loss. Optical loss of various of the described embodiments can be <2 dB/cm. Moreover, compositions described herein may provide higher degree of transparency at wavelengths outside of the absorption bands of the various embodiments herein. In addition, compositions described herein can also exhibit exceptionally high thermal stability, characterized by a decomposition temperature greater than 250 C, as well as high photostability, measured by the degradation of photo-optic materials under broadband light and ambient conditions.

The second-order hyperpolarizability (γ) or third-order susceptibility (χ⁽³⁾), are the normal measures of third-order NLO activity. While there are several methods used to measure these properties, degenerate four-wave mixing (DFWM) is very common. See C. W. Thiel, “Four-wave Mixing and Its Applications,” www.physics.montana.edu.students.thiel.docs/FWMixing.pdf, the entire contents of which are hereby incorporated herein by reference. Referring to Published U.S. Patent Application No. US 2012/0267583A1, the entire contents of which are incorporated herein by reference, a method of evaluating third-order NLO properties of thin films, known in the art as Degenerate Four Wave Mixing (DFWM), can be used. In FIG. 4 of US 2012/0267583A1, Beams 1 and 2 are picosecond, coherent pulses, absorbed by the NLO film deposited on a glass substrate. Beam 3 is a weaker, slightly delayed beam at the same wavelength as Beams 1 and 2. Beam 4 is the resulting product of the wave mixing, diffracted off of the transient holographic grating, produced by interferences of beams 1 and 2 in the NLO material of the film. Beam 3 can be a “control” beam at a telecom wavelength which produces a “signal” beam at a frequency not absorbed by the NLO material.

Nonlinear optical chromophores suitable for use in accordance with the various embodiments of the invention include those having the general formula (I):

D-II-A (I)

wherein D represents an organic electron-donating group; A represents an organic electron-accepting group having an electron affinity greater than the electron affinity of D; and II represents a II-bridge between A and D. The terms electron-donating group (donor or “D”), II-bridge (bridging group or “II”), and electron-accepting group (acceptor or “A”), and general synthetic methods for forming D-II-A chromophores in accordance with the various embodiments described herein are known in the art, for example as described in U.S. Pat. Nos. 5,670,091, 5,679,763, 6,090,332, and 6,716,995, and U.S. patent application Ser. No. 17/358,960, filed on Jun. 25, 2021, the entire contents of each of which is incorporated herein by reference.

An acceptor is an atom or group of atoms that has a low reduction potential, wherein the atom or group of atoms can accept electrons from a donor through a II-bridge. The acceptor (A) has a higher electron affinity than does the donor (D), so that, at least in the absence of an external electric field, the chromophore is generally polarized in the ground state, with relatively more electron density on the acceptor (D). Typically, an acceptor group contains at least one electronegative heteroatom that is part of a pi bond (a double or triple bond) such that a resonance structure can be drawn that moves the electron pair of the pi bond to the heteroatom and concomitantly decreases the multiplicity of the pi bond (i.e., a double bond is formally converted to single bond or a triple bond is formally converted to a double bond) so that the heteroatom gains formal negative charge. The heteroatom may be part of a heterocyclic ring. Exemplary acceptor groups include but are not limited to —NO₂, —CN, —CHO, COR, CO₂R, —PO(OR)₃, —SOR, —SO₂R, and —SO₃R where R is alkyl, aryl, or heteroaryl. The total number of heteroatoms and carbons in an acceptor group is about 30, and the acceptor group may be substituted further with alkyl, aryl, and/or heteroaryl.

Suitable electron-accepting groups “A” (also referred to in the literature as electron-withdrawing groups) for nonlinear optical chromophores that can be used in accordance with the various embodiments of the present invention include those described in published U.S. Patent Applications: US 2007/0260062; US 2007/0260063; US 2008/0009620; US 2008/0139812; US 2009/0005561; US 2012/0267583A1 (collectively referred to as “the prior publications”), each of which is incorporated herein by reference in its entirety; and in U.S. Pat. Nos. 6,584,266; 6,393,190; 6,448,416; 6,44,830; 6,514,434; 5,044,725; 4,795,664; 5,247,042; 5,196,509; 4,810,338; 4,936,645; 4,767,169; 5,326,661; 5,187,234; 5,170,461; 5,133,037; 5,106,211; and 5,006,285; each of which is also incorporated herein by reference in its entirety.

In nonlinear optical chromophores suitable for use in accordance with various embodiments of the present invention, suitable electron-accepting groups can include those according to general formula (A^{a}):

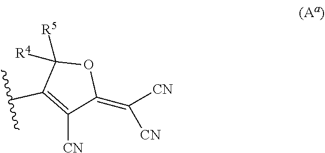

wherein R⁴ and R⁵ each independently represents a moiety selected from the group consisting of H, substituted or unsubstituted C₁-C₁₀ alkyl, substituted or unsubstituted C₂-C₁₀ alkenyl, substituted or unsubstituted C₂-C₁₀ alkynyl, substituted or unsubstituted aryl, substituted or unsubstituted alkylaryl, substituted or unsubstituted carbocyclic, substituted or unsubstituted heterocyclic, substituted or unsubstituted cyclohexyl, and (CH₂)ₙ—O—(CH₂)ₙ where n is 1-10. As used herein,

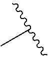

represents a point of bonding to another portion of a larger molecular structure. In various preferred embodiments, one or both of R⁴ and R⁵ represent a halogen-substituted moiety. Halogen-substituted may refer to mono-, di-, tri- and higher degrees of substitution. In various embodiments, one of R⁴ and R⁵ represent a halogen-substituted alkyl moiety and the other represents an aromatic moiety. In various embodiments, one of R⁴ and R⁵ represent a halogen-substituted aromatic moiety and the other represents an alkyl moiety. In various embodiments, the electron-accepting group can be:

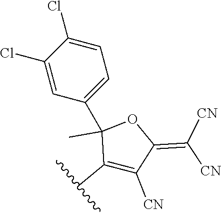 [SMILES: *C1=C(C#N)C(=C(C#N)C#N)OC1(C)c1ccc(Cl)c(Cl)c1]

In various embodiments, the electron-accepting group can be:

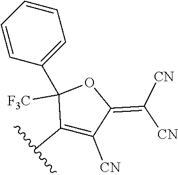 [SMILES: *C1=C(C#N)C(=C(C#N)C#N)OC1(c1ccccc1)C(F)(F)F]

In various embodiments, the electron-accepting group can be:

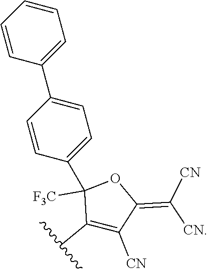 [SMILES: *C1=C(C#N)C(=C(C#N)C#N)OC1(c1ccc(-c2ccccc2)cc1)C(F)(F)F]

In general, the choice of electron-accepting group may match the strength of electron-donating group. The overall strength between electron-donating group and electron-accepting group may not be too weak or too strong. If the overall strength is too weak, the λₘₐₓ may be blue shifted with a lower r₃₃. If the overall strength is too strong, the λₘₐₓ may be red shifted with a lower r₃₃ and more lossy. The choice of R⁴ and R⁵ groups in the electron-accepting group may also depend on the volume of the acceptor moiety to isolate. A better isolation for the acceptor moiety may bring better electro-optics property.

A donor includes an atom or group of atoms that has a low oxidation potential, wherein the atom or group of atoms can donate electrons to an acceptor “A” through a II-bridge. The donor (D) has a lower electron affinity than does the acceptor (A), so that, at least in the absence of an external electric field, the chromophore is generally polarized, with relatively less electron density on the donor (D). Typically, a donor group contains at least one heteroatom that has a lone pair of electrons capable of being in conjugation with the p-orbitals of an atom directly attached to the heteroatom such that a resonance structure can be drawn that moves the lone pair of electrons into a bond with the p-orbital of the atom directly attached to the heteroatom to formally increase the multiplicity of the bond between the heteroatom and the atom directly attached to the heteroatom (i.e., a single bond is formally converted to double bond, or a double bond is formally converted to a triple bond) so that the heteroatom gains formal positive charge. The p-orbitals of the atom directly attached to the heteroatom may be vacant or part of a multiple bond to another atom other than the heteroatom. The heteroatom may be a substituent of an atom that has pi bonds or may be in a heterocyclic ring. Exemplary donor groups include but are not limited to R₂N— and RₙX¹—, where R is alkyl, aryl or heteroaryl, X¹ is O, S, P, Se, or Te, and n is 1 or 2. The total number of heteroatoms and carbons in a donor group may be about 30, and the donor group may be substituted further with alkyl, aryl, or heteroaryl.

In nonlinear optical chromophores suitable for use in accordance with various embodiments of the present invention, suitable electron-donating groups can include the following indolizine donor group (D^{a}):

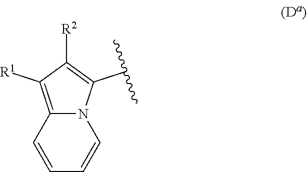

wherein R¹ in the indolizine electron-donating group represents a moiety selected from the group consisting of H, substituted or unsubstituted C₂-C₁₀ alkyl, substituted or unsubstituted C₂-C₁₀ alkenyl, and substituted or unsubstituted C₂-C₁₀ alkynyl, wherein R² in the indolizine electron-donating group represents a moiety selected from the group consisting of substituted monocyclic or unsubstituted aryl, substituted or unsubstituted polycyclic aryl, substituted or unsubstituted alkylaryl, substituted or unsubstituted carbocyclic, substituted or unsubstituted heterocyclic, and substituted or unsubstituted cyclohexyl. The selection of R¹ and R² groups to isolate the nitrogen in the indolizine donor moiety plays an important role in achieving good characters in the nonlinear optical chromophore. Selected R¹ and R² groups on the indolizine donor group may reduce the chromophore-chromophore interaction, since less chromophore-chromophore interaction contributes to better loading limitations, better conductivity, and better characters against dielectric breakdown. Selected R¹ and R² groups on the indolizine donor group may reduce charge transfer and minimize II-bond stacking, since less charge transfer and minimized II-bond stacking contributes to less counterproductive to poling and better solubility.

In various embodiments, the electron-donating group can be:

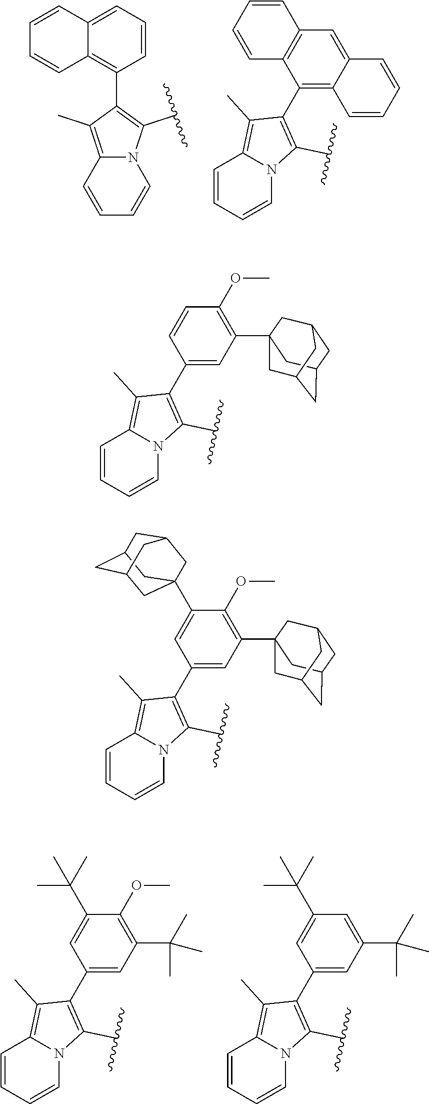

In various embodiments, the electron-donating group can include one or more substituents on the six-member ring of the indolizine structure:

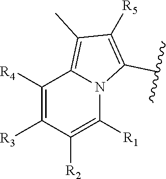

wherein R¹, R², R³ and R⁴ each independently represents a moiety selected from the group consisting of H, heteroatoms, substituted or unsubstituted C₁-C₁₀ alkyl, substituted or unsubstituted C₂-C₁₀ alkenyl, substituted or unsubstituted C₂-C₁₀ alkynyl, substituted or unsubstituted aryl, substituted or unsubstituted alkylaryl, substituted or unsubstituted carbocyclic, substituted or unsubstituted heterocyclic, substituted or unsubstituted cyclohexyl, and/or (CH₂)ₙ—O—(CH₂)ₙ where n is 1-10, and wherein R₅ represents a moiety selected from the group consisting of H and/or substituted or unsubstituted aryl. For example, R₅ may represent a phenyl substituent.

In various examples, the electron-donating group can include a five-member or six-member ring-locked indolizine structure:

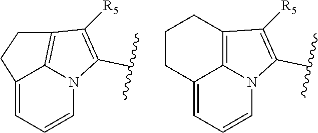

wherein R₅ represents a moiety selected from the group consisting of H and/or substituted or unsubstituted aryl. For example, R₅ may represent a phenyl substituent.

A “II-bridge” includes an atom or group of atoms through which electrons may be delocalized from an electron donor (defined above) to an electron acceptor (defined above) through the orbitals of atoms in the bridge. Typically, the orbitals will be p-orbitals on double (sp²) or triple (sp) bonded carbon atoms such as those found in alkenes, alkynes, neutral or charged aromatic rings, and neutral or charged heteroaromatic ring systems. Additionally, the orbitals may be p-orbitals on atoms such as boron or nitrogen. Additionally, the orbitals may be p, d or f organometallic orbitals or hybrid organometallic orbitals. The atoms of the bridge that contain the orbitals through which the electrons are delocalized are referred to here as the “critical atoms.” The number of critical atoms in a bridge may be a number from 1 to about 30. The critical atoms may be substituted with an organic or inorganic group. The substituent may be selected with a view to improving the solubility of the chromophore in a polymer matrix, to enhance the stability of the chromophore, or for other purposes.

Suitable bridging groups (II) for nonlinear optical chromophores according to general formula (I) can include those described in U.S. Pat. Nos. 6,584,266; 6,393,190; 6,448,416; 6,44,830; 6,514,434; each of which is also incorporated herein by reference in its entirety.

In various embodiments, bridging groups (II) for nonlinear optical chromophores according to general formula (I) can include those of the general formula (II^{a}):

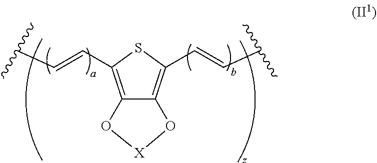

wherein X represents a substituted or unsubstituted, branched or unbranched C₂-C₄ diyl moiety; wherein each a and b independently represents an integer of 0 to 3; and z represents an integer of 1 to 3. In various embodiments wherein a or b in general formula (II¹) is 1, that carbon-carbon double bond in the formula can be replaced with a carbon-carbon triple bond. Alternatively, in various embodiments, bridging groups (II) for nonlinear optical chromophores according to general formula (I) can include those of the general formula (II²):

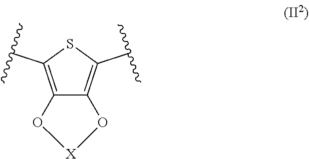

wherein X represents a substituted or unsubstituted, branched or unbranched C₂-C₄ diyl moiety. In various embodiments wherein one or more diamondoid groups is covalently attached to a bridging group according to general formulae II¹ or II², the one or more diamondoid groups may be bound, for example, to the sulfur or oxygen atoms of the thiophene group or to one or more carbon atoms in X through an ether or thioether linkage.

In various embodiments, bridging groups (II) for nonlinear optical chromophores according to general formula (I) can include those of the general formula (II³):

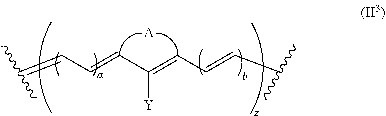

wherein each Y independently represents: a diamondoid-containing group covalently bound to the bridging group through any of the various linkages described herein below including but not limited to ether and thioether linkages; or each Y may represent a hydrogen, an alkyl group, aryl group, sulfur or oxygen linked akyl or aryl group, or a branched or unbranched, optionally heteroatom-containing C₁-C₄ substituent; wherein each a and b independently represents an integer of 0 to 3; z represents an integer of 1 to 3; and wherein each arc A independently represents a substituted or unsubstituted C₂-C₄ alkyl group, which together with the carbon bearing the Y substituent and its two adjacent carbon atoms forms a cyclic group. Substituted or unsubstituted C₂-C₄ alkyl groups which constitute arc A may include 1 to 4 hydrogen substituents each comprising a moiety selected from the group consisting of substituted or unsubstituted C₁-C₁₀ alkyl, substituted or unsubstituted C₂-C₁₀ alkenyl, substituted or unsubstituted C₂-C₁₀ alkynyl, substituted or unsubstituted aryl, substituted or unsubstituted alkylaryl, substituted or unsubstituted carbocyclic, substituted or unsubstituted heterocyclic, substituted or unsubstituted cyclohexyl, and (CH₂)ₙ—O—(CH₂)ₙ where n is 1-10. In various embodiments, z represents 1. In various embodiments, the electron-donating group or electron-accepting group can include one or more covalently bound diamondoid groups, and Y in general formula II³ may represent any of the above substituents. In various embodiments, a chromophore may include an electron-donating group including one or more covalently linked diamondoid groups, preferably adamantyl, and the bridging group may include an isophorone group in accordance with general formula II³ wherein Y represent an aryl thioether substituent.

In various embodiments, bridging groups (II) for nonlinear optical chromophores according to general formula (I) can include those of the general formula (II⁴):

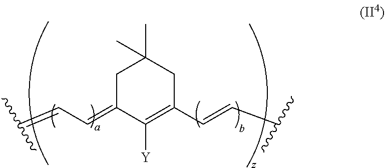

wherein each Y independently represents: a diamondoid-containing group covalently bound to the bridging group through any of the various linkages described herein below including but not limited to ether and thioether linkages; or each Y may represent a hydrogen, an alkyl group, aryl group, sulfur or oxygen linked alkyl or aryl group, an aryl group (optionally bearing a diamondoid group) linked directly by a carbon-carbon bond (e.g., adamantly anisole), a halogen, a halogenated alkyl group, a halogenated aryl group, or a branched or unbranched, optionally heteroatom-containing C₁-C₄ substituent; wherein each a and b independently represents an integer of 0 to 3; and z represents an integer of 1 to 3. In various embodiments, the electron-donating group or electron-accepting group can include one or more covalently bound diamondoid groups, and Y in general formula II⁴ may represent any of the above substituents. In various embodiments, a chromophore may include an electron-donating group including one or more covalently linked diamondoid groups, preferably adamantyl, and the bridging group may include an isophorone group in accordance with general formula II⁴ wherein Y represent an aryl thioether substituent. In various embodiments, each of the geminal methyl groups on the isophorone bridge of the general formula II⁴ can instead independently represent a moiety selected from the group consisting of substituted or unsubstituted C₁-C₁₀ alkyl, substituted or unsubstituted C₂-C₁₀ alkenyl, substituted or unsubstituted C₂-C₁₀ alkynyl, substituted or unsubstituted aryl, substituted or unsubstituted alkylaryl, substituted or unsubstituted carbocyclic, substituted or unsubstituted heterocyclic, substituted or unsubstituted cyclohexyl, halogens, halogenated alkyl groups (e.g., —CF₃), halogenated aryls and heteroaryl groups (e.g., pentafluorothiophenol), and (CH₂)ₙ—O—(CH₂)ₙ where n is 1-10.

In nonlinear optical chromophores suitable for use in accordance with various embodiments of the present invention, suitable bridging groups (II) can include the following structure (II^{a}):

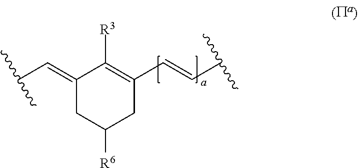

wherein R³ represents a moiety selected from the group consisting of substituted or unsubstituted aryl, substituted or unsubstituted alkylaryl, substituted or unsubstituted carbocyclic, substituted or unsubstituted heterocyclic, and substituted or unsubstituted cyclohexyl; wherein R⁶ represents a moiety selected from the group consisting of H, halogen molecule, substituted or unsubstituted C₂-C₁₀ alkyl, substituted or unsubstituted C₂-C₁₀ alkenyl, and substituted or unsubstituted C₂-C₁₀ alkynyl, substituted or unsubstituted aryl, substituted or unsubstituted alkylaryl, substituted or unsubstituted carbocyclic, substituted or unsubstituted heterocyclic, and substituted or unsubstituted cyclohexyl; and wherein a is an integer of 1 or 2.

The choice of R³ and R⁶ groups may rest on the thermal stability and optical stability of the bridging groups. In other words, a bridging group without good thermal and optical stability may not achieve the desired electro-optic effect.

For example, bridging groups (II) for nonlinear optical chromophores according to general formula (I) can include:

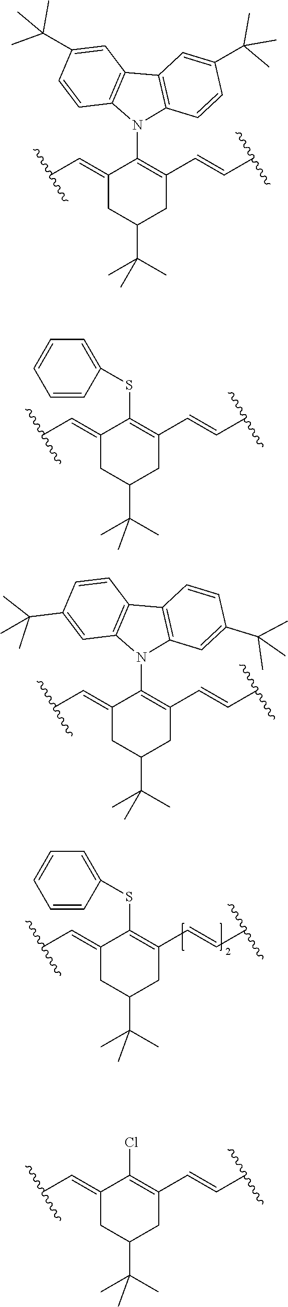

For example, bridging groups (II) for nonlinear optical chromophores according to general formula (I) can include:

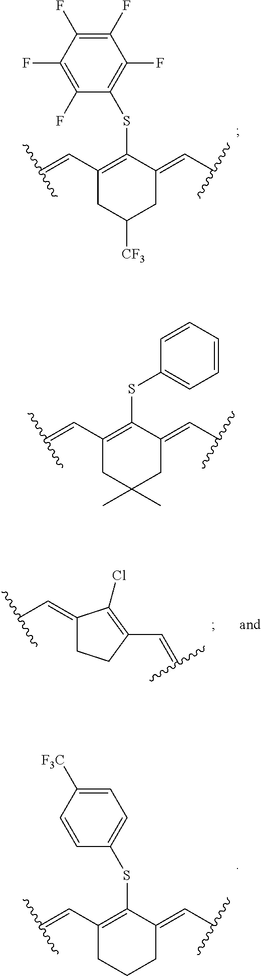

In various embodiments, the nonlinear optical chromophore with indolizine donor group may include, for example:

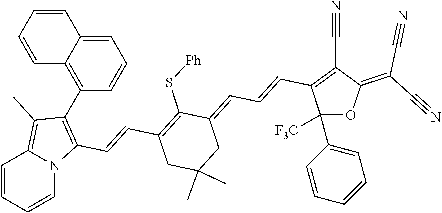 [SMILES: Cc1c(-c2cccc3ccccc23)c(/C=C/C2=C(Sc3ccccc3)C(=C/C=C/C3=C(C#N)C(=C(C#N)C#N)OC3(c3ccccc3)C(F)(F)F)/CC(C)(C)C2)n2ccccc12]

Indolizine donor groups suitable for use in the various embodiments of the present invention may be substituted or unsubstituted, including hydro and alkyl substituents, aryl substituents and combination thereof. The substituent moieties in the nonlinear optical chromophores with indolizine donor groups may be important for isolation. The isolation may reduce chromophore-chromophore interaction. The isolation may reduce charge transfer between molecules. The isolation may increase maximum applied voltage, which may contribute to more optimal poling and higher r₃₃.

On one hand, nonlinear optical chromophores with indolizine donor group in accordance with various embodiments of the present invention may be a short chromophore with indolizine donor or a long chromophore with indolizine donor group. The short chromophore with indolizine donor may have three conjugated double bonds as II-bridge between the acceptor and the donor. The short chromophore may only be feasible if there is a strong donor (e.g., indolizine donor) that provides a red shifted maximum absorption (λₘₐₓ). The short chromophore may therefore provide a narrow λₘₐₓ and reduce the loss of light in a device. On the other hand, nonlinear optical chromophores with indolizine donor group in accordance with various embodiments of the present invention may be a long chromophore with indolizine donor group. The long chromophore with indolizine donor may have four or more conjugated double bonds as II-bridge between the acceptor and the donor.

What is claimed is:

1. A nonlinear optical chromophore of the general formula (I):
D-II-A (I)
wherein D represents an organic indolizine electron-donating group having the following formula (D^{a}): 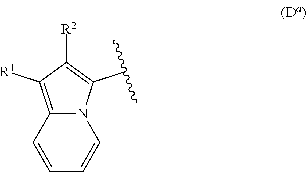
wherein R¹ in the indolizine electron-donating group represents a moiety selected from the group consisting of H, substituted or unsubstituted C₂-C₁₀ alkyl, substituted or unsubstituted C₂-C₁₀ alkenyl, and substituted or unsubstituted C₂-C₁₀ alkynyl, wherein R² in the indolizine electron-donating group represents a moiety selected from the group consisting of substituted monocyclic aryl, substituted or unsubstituted polycyclic aryl, substituted or unsubstituted alkylaryl, substituted or unsubstituted carbocyclic, substituted or unsubstituted heterocyclic, and substituted or unsubstituted cyclohexyl;
wherein A represents an organic electron-accepting group having an electron affinity greater than the electron affinity of D; and II represents a II-bridge between A and D, wherein the II-bridge comprises a carbon chain covalently bound to and separating A and D.

2. The nonlinear optical chromophore according to claim 1, wherein the carbon chain length between A and D is 6 to 8 carbon atoms.

3. The nonlinear optical chromophore according to claim 1, wherein the carbon chain length between A and D is 4 carbon atoms.

4. The nonlinear optical chromophore according to claim 1, wherein the carbon chain length between A and D is 10 carbon atoms.

5. The nonlinear optical chromophore according to claim 2, wherein the Amax value is in a range of 670 nm and 875 nm.

6. The nonlinear optical chromophore according to claim 2, wherein the Δλₘₐₓ value is equal to or smaller than 130 nm.

7. The nonlinear optical chromophore according to claim 2, wherein II represents a II-bridge between A and D having the following formula (II^{a}): 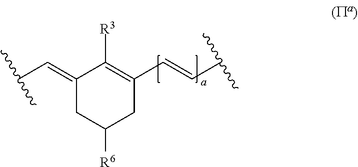
wherein R³ represents a moiety selected from the group consisting of substituted or unsubstituted aryl, substituted or unsubstituted alkylaryl, substituted or unsubstituted carbocyclic, substituted or unsubstituted heterocyclic, and substituted or unsubstituted cyclohexyl; wherein R⁶ represents a moiety selected from the group consisting of H, halogen molecule, substituted or unsubstituted C₂-C₁₀ alkyl, substituted or unsubstituted C₂-C₁₀ alkenyl, and substituted or unsubstituted C₂-C₁₀ alkynyl, substituted or unsubstituted aryl, substituted or unsubstituted alkylaryl, substituted or unsubstituted carbocyclic, substituted or unsubstituted heterocyclic, and substituted or unsubstituted cyclohexyl; and wherein a is an integer of 1 or 2.

8. The nonlinear optical chromophore according to claim 1, wherein A represents an electron-accepting group of the general formula (A^{a}): 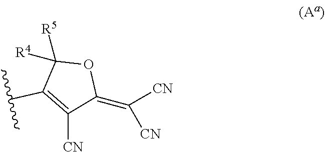
wherein R⁴ and R⁵ each independently represents a moiety selected from the group consisting of H, substituted or unsubstituted C₁-C₁₀ alkyl, substituted or unsubstituted C₂-C₁₀ alkenyl, substituted or unsubstituted C₂-C₁₀ alkynyl, substituted or unsubstituted aryl, substituted or unsubstituted alkylaryl, substituted or unsubstituted carbocyclic, substituted or unsubstituted heterocyclic, substituted or unsubstituted cyclohexyl, and (CH₂)ₙ—O—(CH₂)ₙ where n is 1-10.

9. The nonlinear optical chromophore according to claim 1, wherein the nonlinear chromophore has the general formula (C′):

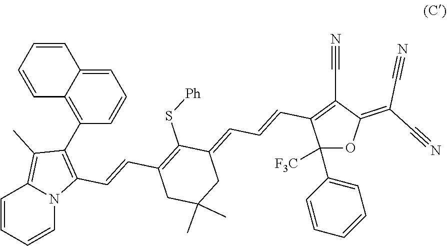

10. A nonlinear optical chromophore of the general formula (I):
D-II-A (I)
wherein D represents an organic indolizine electron-donating group having the following formula: 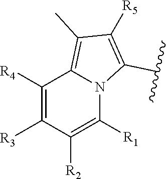
wherein R¹, R², R³ and R⁴ each independently represents a moiety selected from the group consisting of heteroatoms, substituted or unsubstituted C₁-C₁₀ alkyl, substituted or unsubstituted C₂-C₁₀ alkenyl, substituted or unsubstituted C₂-C₁₀ alkynyl, substituted or unsubstituted aryl, substituted or unsubstituted alkylaryl, substituted or unsubstituted carbocyclic, substituted or unsubstituted heterocyclic, substituted or unsubstituted cyclohexyl, or (CH₂)ₙ—O—(CH₂), where n is 1-10, and wherein R₅ represents a moiety selected from the group consisting of H or substituted or unsubstituted aryl;
wherein A represents an organic electron-accepting group having an electron affinity greater than the electron affinity of D; and/7 represents a II-bridge between A and D, wherein the/7-bridge comprises a carbon chain covalently bound to and separating A and D.

11. The nonlinear optical chromophore according to claim 10, wherein R₅ represents a phenyl moiety.

12. The nonlinear optical chromophore according to claim 10, wherein II represents a II-bridge between A and D having the following formula (II^{a}): 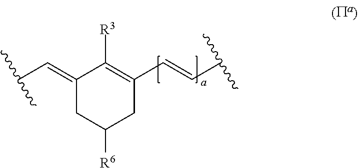
wherein R³ represents a moiety selected from the group consisting of substituted or unsubstituted aryl, substituted or unsubstituted alkylaryl, substituted or unsubstituted carbocyclic, substituted or unsubstituted heterocyclic, and substituted or unsubstituted cyclohexyl; wherein R⁶ represents a moiety selected from the group consisting of H, halogen molecule, substituted or unsubstituted C₂-C₁₀ alkyl, substituted or unsubstituted C₂-C₁₀ alkenyl, and substituted or unsubstituted C₂-C₁₀ alkynyl, substituted or unsubstituted aryl, substituted or unsubstituted alkylaryl, substituted or unsubstituted carbocyclic, substituted or unsubstituted heterocyclic, and substituted or unsubstituted cyclohexyl; and wherein a is an integer of 1 or 2.

13. The nonlinear optical chromophore according to claim 10, wherein A represents an electron-accepting group of the general formula (A^{a}): 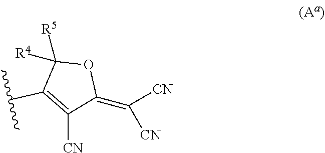
wherein R⁴ and R⁵ each independently represents a moiety selected from the group consisting of H, substituted or unsubstituted C₁-C₁₀ alkyl, substituted or unsubstituted C₂-substituted or unsubstituted alkylaryl, substituted or unsubstituted carbocyclic, substituted or unsubstituted heterocyclic, substituted or unsubstituted cyclohexyl, and (CH₂)ₙ—O—(CH₂) n where n is 1-10.

14. A nonlinear optical chromophore of the general formula (I):
D-II-A (I)
wherein D represents an organic indolizine electron-donating group having one of the following formulas: 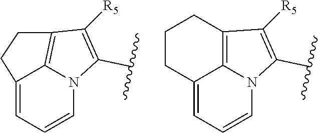
wherein R₅ represents a moiety selected from the group consisting of H or substituted or unsubstituted aryl;
wherein A represents an organic electron-accepting group having an electron affinity greater than the electron affinity of D; and II represents a II-bridge between A and D, wherein the II-bridge comprises a carbon chain covalently bound to and separating A and D.

15. The nonlinear optical chromophore according to claim 14, wherein R₅ represents a phenyl moiety.

16. The nonlinear optical chromophore according to claim 14, wherein II represents a II-bridge between A and D having the following formula (II^{a}): 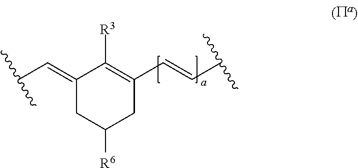
wherein R³ represents a moiety selected from the group consisting of substituted or unsubstituted aryl, substituted or unsubstituted alkylaryl, substituted or unsubstituted carbocyclic, substituted or unsubstituted heterocyclic, and substituted or unsubstituted cyclohexyl; wherein R⁶ represents a moiety selected from the group consisting of H, halogen molecule, substituted or unsubstituted C₂-C₁₀ alkyl, substituted or unsubstituted C₂-C₁₀ alkenyl, and substituted or unsubstituted C₂-C₁₀ alkynyl, substituted or unsubstituted aryl, substituted or unsubstituted alkylaryl, substituted or unsubstituted carbocyclic, substituted or unsubstituted heterocyclic, and substituted or unsubstituted cyclohexyl; and wherein a is an integer of 1 or 2.

17. The nonlinear optical chromophore according to claim 14, wherein A represents an electron-accepting group of the general formula (A^{a}): 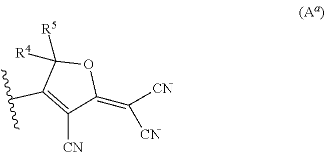
wherein R⁴ and R⁵ each independently represents a moiety selected from the group consisting of H, substituted or unsubstituted C₁-C₁₀ alkyl, substituted or unsubstituted C₂-C₁₀ alkenyl, substituted or unsubstituted C₂-C₁₀ alynyl, substituted or unsubstituted aryl, substituted or unsubstituted alkylaryl, substituted or unsubstituted carbocyclic, substituted or unsubstituted heterocyclic, substituted or unsubstituted cyclohexyl, and (CH₂)ₙ—O—(CH₂)ₙ where n is 1-10.
